(19) United States
(12) Patent Application Publication
(10) Pub. No.: US 2006/0111774 A1
(43) Pub. Date: May 25, 2006

(54) Low noise heart valve prosthesis and method for operation

(76) Inventors: Alexander V. Samkov, Moscow (RU); Naum A. Iofis, Moscow (RU); Andrey V. Agafonov, Moscow (RU)

Correspondence Address: DUANE MORRIS LLP;IP DEPARTMENT, 30 SOUTH 17TH STREET, PHILADELPHIA, PA, 19103-4196, US

(21) Appl. No.: 11/114,587

(22) Filed: Apr. 26, 2005

Related U.S. Application Data
(60) Provisional application No. 60/630,618, filed on Nov. 24, 2004.

(51) Int. Cl. A61F 2/24

(52) U.S. Cl. 623/2.25

(57) ABSTRACT

A heart valve prosthesis having an annular support and an occluder-disc pivotally mounted in a fluid passageway of the annular support, where the annular support includes at least a first axis of symmetry. A pier that projects radially inwardly from an inner surface of the annular support and along the first axis of symmetry. A first arm projects inwardly from the inner surface of the annular support and a second arm that projects inwardly from the inner surface of the annular support so as to be in confronting spaced relation to the first arm thereby defining a second axis of symmetry. The first axis of symmetry and the second axis of symmetry are arcuately off-set from one another. A method of operation of the heart valve that reduces wear in the occluder-disc and noise is also presented.

### CROSS-REFERENCE OF RELATED APPLICATION

This application is related to, and claims priority from provisional patent application Ser. No. 60/630,618, filed Nov. 24, 2004.

### FIELD OF THE INVENTION

The present invention generally relates to prosthetics used in heart surgery, and more particularly to prosthetics used for the replacement of mitral valves of a heart.

### BACKGROUND OF THE INVENTION

It is well known to replace damaged human heart valves with prostheses having movable vanes, flaps or balls which allow blood flow in one direction and prevent blood flow back in the opposite direction. Many different designs based on a variety of concepts are well known in the art. Typically, such prosthetic heart valves include a ring-shaped case having a disc-shaped closure that is installed within the ring-shaped case with the ability of repeatedly opening and closing the throat of the case. Symmetrical top and bottom supports are also often provided to limit the disc-shaped closure's movements when the prosthetic heart valve is opening and closing.

For example, in U.S. Pat. No. 4,240,161, issued to Huffstutler et al., a free floating pivoting disc heart valve is discussed. The disc has a generally arcuate segment configuration positioned so as to present a generally convex surface toward the blood outflow side of the base. A generally concave surface is directed toward the inflow side of the base when viewed in a closed position. Huffstutler et al., suggest that their device avoids problems in the art related to incomplete opening of the valve, excessive pressure losses across the valve, and excessive back flow through the valve during the closing phase.

In U.S. Pat. No. 4,494,253, issued to Bicer, a cardiovascular prosthesis and a method for its manufacture are disclosed. The prosthesis consists of a support having a perimetral groove and an occluding disc which is assembled on the support so as to be freely rotatable. A textile ring is located in the perimetral groove and consists of a tubular fabric. It is suggested by Bicer that due to the quality of the material used for support of the occluding disc, as well as the particular shape of the disc and manufacturing process, the prosthesis presents unusual physical, mechanical and chemical characteristics and superior performance. It is also suggested that the support is practically inert to solutions, acids, or alkalis at room temperature.

In U.S. Pat. No. 4,725,275, issued to Moll, a heart valve is provided that includes a disc that is pivotally mounted within a ring. The disc and ring are arranged so that blood flows through the valve as the disc moves relative to the ring so as to reduce blood clot formations, and the likelihood of thrombosis. By permitting the disc to rotate about its axis and to float within its base, i.e., pivotal, rotational and translatory movements, Moll suggests that the likelihood of blood clot formations at the pivot points of the vane is reduced. Thus, Moll's artificial heart valve provides increased float of the vane to reduce the likelihood of thrombosis, regardless of the position it is located in the heart, namely the mitral, tricuspid, pulmonic or aortic positions.

In U.S. Pat. No. 4,822,355, issued to Bhuvaneshwar, a heart valve assembly is disclosed including a heart valve disc tiltably mounted within a circular ring of metal. A fabric sewing ring is attached to the circular metal ring so as to enable the whole assembly to be stitched to surrounding tissue of the body. Bhuvaneshwar suggests that his heart valve assembly avoids disc wear and maintains proper balance. Bhuvaneshwar 's disc is installed on supporting elements within his ring which extend radially from its internal surface. One supporting element is placed on one side of the disc, and the other two are placed approximately 60° away from each other on the circle of the ring. Although Bhuvaneshwar's valve appears to provide conditions for good blood flow and thrombosis resistance, it appears to also generate a working noise level that tends to disturb patients.

In Soviet inventor's certificate No. SU 1832465, an artificial cardiac valve is disclosed that suggests its structure reduces the level of self-inflicted trauma by dampening a occluding disc. The artificial cardiac valve consists of a housing with a disc-holder located on a toroidal surface of which a plurality of protrusions has been provided to damp a convex-concave occluding disc.

In British Patent No. GB 1089079, a heart valve is disclosed consisting of an angular ring having a cage-like structure projecting from an upstream side. The cage-like structure maintains a variety of different occluding devices, including discs and balls, loosely in place so as to allow for an opening and closing of the valve. The extent and height of the cage-like structure above the ring helps to regulate the opening and closing of the valve during normal heart operation.

In Russian Federation Patent No. RU 2146905, a prosthetic heart valve is disclosed having a circular housing that is possible to rotate for opening or closing a passage section of the housing. An obturating member, such as a disc, and upper and lower stops are provided to impose restrictions upon the movement of the disc when the valve opens and closes. The lower stops are in the form of half-arcs protruding from the internal lateral surfaces of the ring and are arranged so as to form a gap between their free ends. The lower stops are made equal in size and the free ends are at different distances from a transverse axis of symmetry of the ring.

Thus common design goals sought by most prior art heart valve prostheses include the selection of strong, wear proof, chemically inert materials for both the moving and support of the valve. However, irrespective of material selection, each time the valve prosthesis opens and closes the occluding disc makes contact with its supporting elements at the same impact point on the disc's surface. This repeated contact, during the long working life of the valve (ten years or more) has often lead to non-uniform deterioration of the occluding disc's surface and, as a consequence, lead to an irreparable degradation of the working rhythm of the valve or, in some instance the catastrophic failure of the valve. In addition, such prior art prosthetic heart valves produce operational noise at levels which are disturbing to their host.

### SUMMARY OF THE INVENTION

The presented invention provides for the non-uniform deterioration of an occluder-disc's surface as well as lowering the noise output created by the valve during operation. In one embodiment, a heart valve prosthesis is provided that includes an annular support having a fluid passageway with an upstream side and a downstream side and at least a first axis of symmetry. A pier projects radially inwardly from an inner surface of the annular support along the first axis of symmetry. A first arm projects inwardly from an inner surface of the annular support and a second arm projects inwardly from the inner surface of the annular support so as to be in confronting spaced relation to the first arm thereby defining a second axis of symmetry. The first axis of symmetry and the second axis of symmetry are arcuately off-set from one another. An occluder-disc is pivotally mounted in the fluid passageway of the annular support. The heart valve prosthesis according to the invention often has a first axis of symmetry and a second axis of symmetry that are arcuately off-set from one another by an angle of between 1.5° to 4°, and preferably by an angle of between 2° to 3°. The free ends of arms stick out from the first axis of symmetry passing through the pier, at unequal distances, which differ by about 1/80 to about 1/16 of the internal diameter of the annular support.

In another embodiment of the invention, a method for operation of a heart valve prosthesis is provided in which an occluder-disc rotates open within a support ring about a first axis so as to allow blood to flow through a fluid passageway of the support ring. The occluder-disc rotates closed about the first axis while also incrementally rotating about a second axis so as to shift the portions of the occluder-disc that impact the support ring upon closure of the heart valve.

In a further embodiment of the invention, a method for operation of a heart valve prosthesis is provided in which an occluder-disc rotates open within a support ring about a first axis so as to allow blood to flow through a fluid passageway of the support ring. The occluder-disc rotates closed about the first axis so that portions of the occluder-disc impact the support ring upon closure of the heart valve at different times.

In yet a further embodiment of the invention, a method for operation of a heart valve prosthesis is provided in which an occluder-disc rotates open within a support ring about a first axis so as to allow blood to flow through a fluid passageway of the support ring. The occluder-disc rotates closed about the first axis while also incrementally rotating about a second axis so as to shift the portions of the occluder-disc that impact the support ring upon closure of the heart valve and so that the portions of the occluder-disc that impact the support ring upon closure of the heart valve do so at different times.

### BRIEF DESCRIPTION OF THE DRAWINGS

These and other features and advantages of the present invention will be more fully disclosed in, or rendered obvious by, the following detailed description of the preferred embodiments of the invention, which are to be considered together with the accompanying drawings wherein like numbers refer to like parts and further wherein:

FIG. 1 is a perspective view of a low noise heart valve prosthesis formed in accordance with the present invention and positioned within a human heart;

FIG. 2 is an exploded perspective view of the low noise heart valve prosthesis shown in-situ in FIG. 1;

FIG. 3 is a perspective view of the heart valve prosthesis shown in FIG. 2, fully assembled and open;

FIG. 4 is a cross-sectional view of the heart valve prosthesis shown in FIG. 3;

FIG. 5 is a cross-sectional view of the heart valve prosthesis shown in FIG. 4, in a fully closed state;

FIG. 6 is a top plan view of an annular support portion of the heart valve prosthesis shown in FIGS. 1-5, with an occluder-disc removed for clarity of illustration; and

FIG. 7 is a bottom plan view of the annular support shown in FIG. 6.

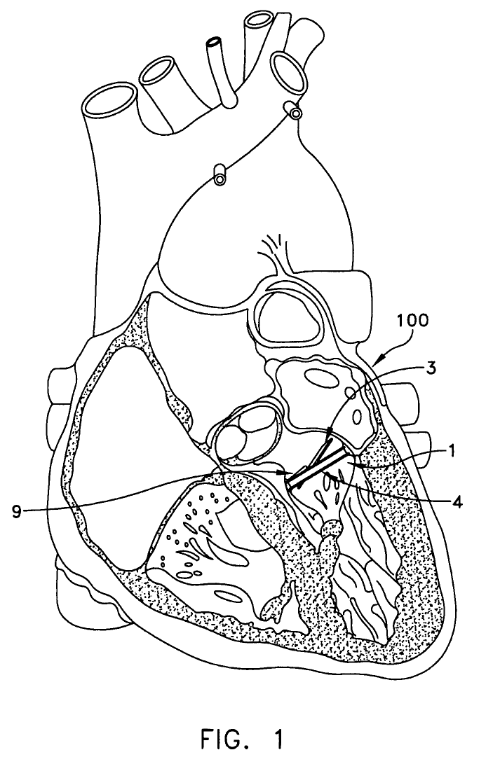

### DETAILED DESCRIPTION OF THE PREFERRED EMBODIMENT

This description of preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom”as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

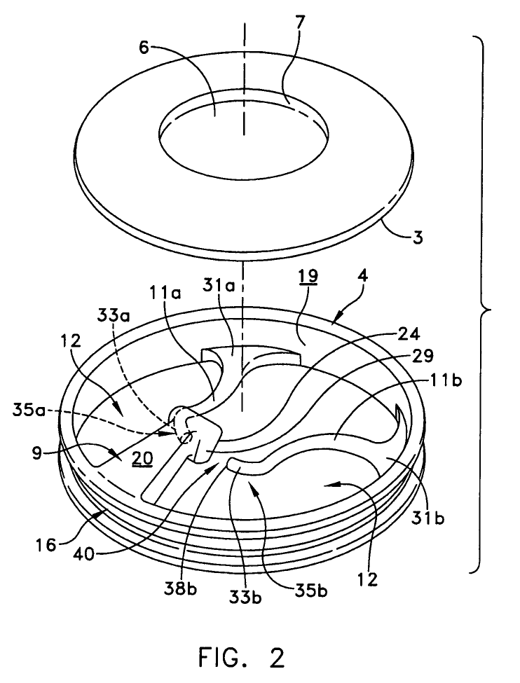

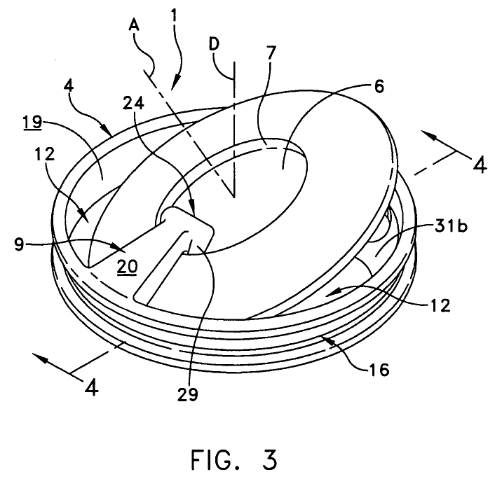

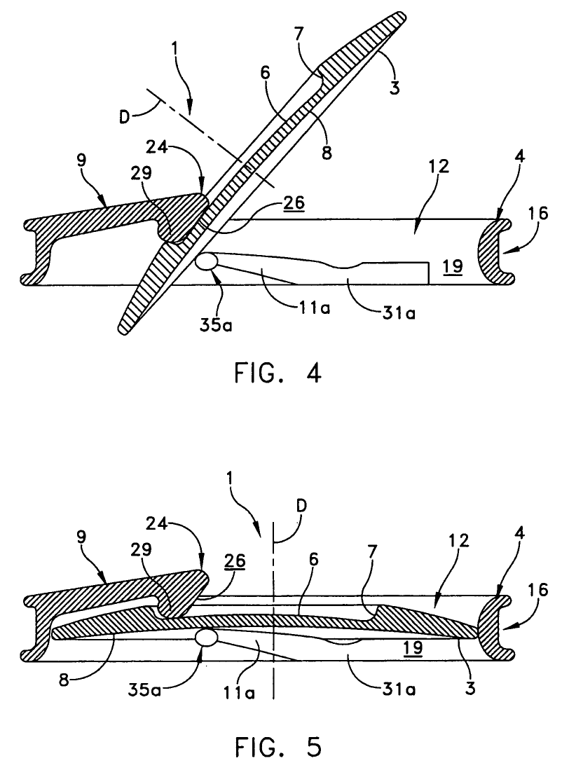

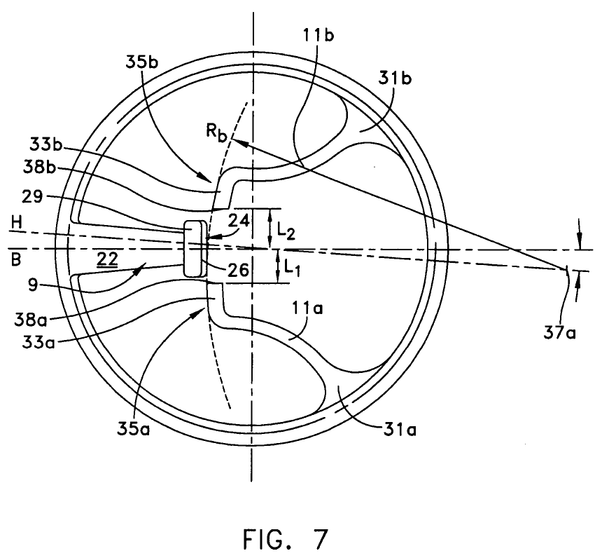

Referring to FIGS. 2-7, a prosthetic heart valve 1 formed in accordance with the present invention includes an occluder-disc 3 and an annular case 4. Occluder-disc 3 has a peripheral shape that closely matches the internal shape of annular case 4, preferably circular, and includes a recessed top surface 6 defined by an annular shoulder wall 7 and a concavely curved bottom surface 8 (FIGS. 2 and 4). Occluder-disc 3 also defines a transverse or diametric axis A that is defined within the plane of the disc and a central, rotational axis D that passes perpendicularly through the center of occluder-disc 3. Axes A and D are normally orthogonal to one another (FIG. 3). Annular case 4 has an upstream side and a downstream side and includes a pier 9 and a pair of arms 11a, 11b that together support occluder-disc 3 which pivots open and closed during operation of prosthetic heart valve 1 about transverse, diametric axis A. More particularly, annular case 4 is often circular in shape so as to comprise a circular symmetry and defines a central opening or fluid passageway 12. An annular channel 16 is defined on an outer surface of annular case 4, and an inner surface 19 of annular case 4 is convexly curved. Pier 9 projects radially inwardly from inner surface 19 so as to form a cantilever, and has a substantially flat, upwardly inclined top surface 20, a recessed, upwardly inclined undersurface 22 (FIG. 7), and a snout 24. An acutely angled outer surface 26 of snout 24 is inclined downwardly toward the confronting portion of inner surface 19 of annular case 4 (FIGS. 4 and 5), and a pivot knob 29 is formed adjacent to recessed undersurface 22. In this way, snout 24 of pier 9 stands proud of annular case 4 on the upstream side, and spaced away from arms 11a, 11b, so as to thereby limit the rotational movement of occluder-disc 3 during valve opening. The angle of acutely inclined outer surface 26 of snout 24 is selected so as to determine the opening angle of occluder-disc 3 during operation of prosthetic heart valve 1.

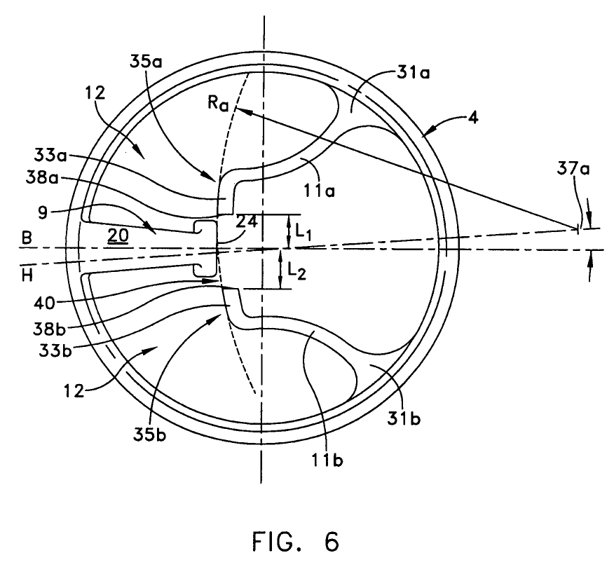

Each arm 11a, 11b projects inwardly from inner surface 19 into opening 12 along a chord of the circle defined by annular case 4 (FIGS. 2 and 6-7) so as to form a curved cantilever or half-arc having a base 31a, 31b located on inner surface 19 and a pivot 33a, 33b located at a free end 35a, 35b. Each pivot 33a, 33b often has a cylindrical cross-sectional shape, but with an outer surface profile that follows a curve defined by a radius emanating from outside annular case 4. Referring to FIGS. 6 and 7, pivot 33a has a curvature defined by a radius Rₐ with a center or point of origin 37a located on an axis of symmetry B of annular case 4 that also passes through a central portion of pier 9, while pivot 33b has a curvature defined by a radius R_{b} with a center or point of origin 37b located on an axis of symmetry H of arms 11a, 11b. In this way, ends 38a, 38b of each pivot 33a, 33b are arranged in spaced-apart confronting relation to one another within central opening 12 thereby forming a gap 40 between pivots 33a, 33b (FIG. 2). Advantageously, arms 11a, 11b are preferably formed in different sizes with their respective ends 38a, 38b being located at different distances from axis of symmetry B of annular case 4. The term “symmetry” refers to a near exact correspondence of form and constituent configuration on opposite sides of a dividing line or plane or about a center or an axis, i.e., an “axis of symmetry.” In the present invention, annular case 4 comprises bilateral symmetry, or two-sidedness, such that it can be divided into symmetrical halves by a vertical plane (a plane containing axis of symmetry B) passing through the middle or center of its ring shape. Pier 9 also comprises bilateral symmetry about axis of symmetry B.

Arms 11a, 11b also define an axis of symmetry H, about which they are symmetric along their full length, except for their ends 38a, 38b. Thus, arms 11a, 11b have substantially near exact correspondence of form and constituent configuration, and are located on opposite sides axis of symmetry H. The distance from end 38a to axis of symmetry B (identified generally at L₁ in FIGS. 6 and 7) is not equal to the distance from end 38b to axis of symmetry B (identified generally at L₂ in FIGS. 6 and 7). It has been found that the optimum difference in distances from each of end 38a, 38b to axis of symmetry B is between about 0.0125 to about 0.0625 times the internal diameter of annular case 4. This relationship holds for the distance between ends 38a, 38b and axis of symmetry H. In this way, axis of symmetry B of annular case 4 passing through the central portion of pier 9, and axis of symmetry H of arms 11a, 11b are arcuately off-set relative to one another about the center of annular case 4 by between about 1.5° to about 4°. A preferred angular off-set is between about 2° to about 3°. Arms 1 1a, 11b limit the rotational movement of occluder-disc 3 (about rotational axis D in FIG. 3) during valve closing. It has been found that when this angular relationship is less then 1.5° occluder-disc 3 may not rotate or pivot about its axis during operation, and when this angular relationship is more than 4°, there is a danger of occluder-disc 3 skewing, rendering prosthetic heart valve 1 in operable.

Prosthetic heart valve 1 operates according to the invention as follows. During opening of prosthetic heart valve 1, occluder-disc 3 pivots upwardly about pivots 33a, 33b until recessed top surface 6 engages inclined outer surface 26 of snout 24, with shoulder wall 7 and pivot knob 29 acting as a pseudo-hinge structure. When fully open, a stream of blood flows outwardly through prosthetic heart valve 1 in a forward direction through heart 100. At the end of the forward movement of the blood stream, the flow reverses causing occluder-disc 3 to pivot back away from pier 9 and toward opening 12. At the end of this pivoting motion, occluder-disc 3 impacts upon portions of arms 11a, 11b and bases 31a, 31b. However, unlike prior art heart valves, occluder-disc 3 does not impact arms 11a, 11b simultaneously, but instead there is a delay caused by the arcuately off-set relationship between axis of symmetry H and axis of symmetry B.

More particularly, during each opening and closing cycle of prosthetic heart valve 1, occluder-disc 3 turns around its rotational axis D by some 3°-6°. Such movement occurs because axis of symmetry B and axis of symmetry H are arcuately off-set by about 1.5° to 4°, preferably by between 2°-3°. Depending upon the direction of arcuate off-set of axis of symmetry H relative to axis of symmetry B, the rotation of occluder-disc 3 about rotational axis D during operation may be clockwise or anticlockwise. Both directions of rotation are equally applicable and do not affect the essence of the invention. In any event, the sound waves generated by the impact of occluder-disc 3 upon portions of arms 11a, 11b along with the rotating occluder-disc 3 and bases 31a, 31b are divided by their power and fluctuation frequency, depending upon the length of arms 11a, 11b. In this way, arms 11a, 11b absorb a portion of the kinetic energy of occluder-disc 3, and thereby weaken the reverberating vibrations in prosthetic heart valve 1 resulting from cyclical impacts of occluder-disc 3 upon arms 11a, 11b. This in turn reduces the acoustic fluctuations perceived by the person relying upon prosthetic heart valve 1 for near normal heart function.

Thus, a prosthetic heart valve 1 formed in accordance with the present invention provides a lowered perceptible acoustic signature, with a uniform degree of deterioration of occluder-disc 3 during long-term operation. Advantageously, occluder-disc 3 impacts both cantilevered arms 11a, 11b non-simultaneously, i.e., at different times. The short delay between impacts with arms 11a, 11b results from occluder-disc 3 approaching arms 11a, 11b in a rotating, non-parallel manner during the closing phase of operation of prosthetic heart valve 1. In other words, the outer surfaces of arms 11a, 11b that face inwardly curved bottom surface 8 of occluder-disc 3 are arranged at an angle, thereby causing multiple impacts, rather than one simultaneous or mono-impact between occluder-disc 3 and arms 11a, 11b. In this way, each impact is essentially weaker than a mono-impact and has a different spectrum of acoustic frequencies.

It is to be understood that the present invention is by no means limited only to the particular constructions herein disclosed and shown in the drawings, but also comprises any modifications or equivalents within the scope of the claims.

What is claimed is:

1. A heart valve prosthesis comprising:
an annular support having a fluid passageway with an upstream side and a downstream side and at least a first axis of symmetry;
a pier projecting radially inwardly from said inner surface of said annular support and along said first axis of symmetry;
a first arm projecting inwardly from an inner surface of said annular support and a second arm projecting inwardly from said inner surface of said annular support so as to be in confronting spaced relation to said first arm thereby defining a second axis of symmetry, wherein said first axis of symmetry and said second axis of symmetry are arcuately off-set from one another; and
an occluder-disc pivotally mounted between said pier and said first and second arms in said fluid passageway of said annular support.

2. A heart valve according to claim 1 wherein said first axis of symmetry and said second axis of symmetry are arcuately off-set from one another by between about 1.5° and about 4°.

3. A heart valve according to claim 1 wherein said first axis of symmetry and said second axis of symmetry are arcuately off-set from one another by between about 2° and about 3°.

4. A heart valve according to claim 1 wherein said first axis of symmetry and said second axis of symmetry are arcuately off-set from one another by about 2°.

5. A heart valve according to claim 1 wherein said first axis of symmetry and said second axis of symmetry are arcuately off-set from one another by about 3°.

6. A heart valve according to claim 1 wherein said occluder-disc defines a recessed top surface defined by a shoulder wall and a concavely curved bottom surface.

7. A heart valve according to claim 1 wherein said pier forms a cantilever with a substantially flat, upwardly inclined top surface, a recessed upwardly inclined undersurface, and a snout.

8. A heart valve according to claim 7 wherein said snout includes an acutely angled outer surface that is inclined downwardly toward a confronting portion of said inner surface of said annular support, and further includes a pivot knob formed adjacent to said recessed undersurface.

9. A heart valve according to claim 1 wherein said snout of said pier stands proud of said annular support and spaced away from said arms so as to thereby limit the rotational movement of said occluder-disc about a transverse axis during valve opening.

10. A heart valve according to claim 1 wherein said annular support comprises a circular symmetry.

11. A heart valve according to claim 1 wherein said first and said second arms each project inwardly from an inner surface of said annular support along a chord of the circle defined by said annular support so as to form curved cantilevers.

12. A heart valve according to claim 1 wherein said first and said second arms each include a pivot located at a free end and having an outer surface profile that follows a curve defined by a radius emanating from a point outside said annular support.

13. A heart valve according to claim 12 wherein said pivot of said first arm has a curvature defined by a radius having a point of origin located on said first axis of symmetry

14. A heart valve according to claim 12 wherein said pivot of said second arm has a curvature defined by a radius having a point of origin located on said second axis of symmetry.

15. A heart valve according to claim 12 wherein each of said pivots is arranged in spaced-apart confronting relation to one another within said fluid passageway thereby forming a gap between them such that said free ends of said pivots are located at different distances from said first axis of symmetry.

16. A heart valve according to claim 12 wherein said free end of said first arm is arranged in spaced-apart confronting relation to said free end of said second arm thereby forming a gap between said free ends such that the distance from said free end of said first arm to said first axis of symmetry is different than the distance from said free end of said second arm to said first axis of symmetry.

17. A heart valve according to claim 16 wherein the difference in said distances from said free ends of said arms to said first axis of symmetry is in the range from about between about 0.0125 to about 0.0625 times an internal diameter of said annular support.

18. A heart valve according to claim 12 wherein said free end of said first arm is arranged in spaced-apart confronting relation to said free end of said second arm thereby forming a gap between said free ends such that the distance from said free end of said first arm to said second axis of symmetry is different than the distance from said free end of said second arm to said second axis of symmetry.

19. A heart valve according to claim 18 wherein the difference in said distances from said free ends of said arms to said second axis of symmetry is in the range from about between about 0.0125 to about 0.0625 times an internal diameter of said annular support.

20. A heart valve prosthesis comprising:
a support ring having a central opening and at least a first axis of symmetry;
a pier projecting radially inwardly from an inner surface of said support ring and symmetrically arranged about said first axis of symmetry;
a first arm projecting inwardly from an inner surface of said support ring and a second arm projecting inwardly from said inner surface of said support ring so as to be in spaced relation to said first arm thereby defining a second axis of symmetry, wherein said first axis of symmetry and said second axis of symmetry are arcuately off-set from one another by between about 2° and about 3°; and
an occluder-disc pivotally mounted between said pier and said first and second arms in said central opening of said support ring.

21. A heart valve according to claim 20 wherein said first and said second arms each include a pivot located at a free end and having an outer surface profile that follows a curve defined by a radius emanating from a point outside said support ring.

22. A heart valve according to claim 21 wherein said pivot of said first arm has a curvature defined by a radius having a point of origin located on said first axis of symmetry

23. A heart valve according to claim 21 wherein said pivot of said second arm has a curvature defined by a radius having a point of origin located on said second axis of symmetry.

24. A heart valve according to claim 21 wherein each of said pivots is arranged in spaced-apart relation to one another thereby forming a gap between them such that said free ends of said pivots are located at different distances from said first axis of symmetry.

25. A heart valve according to claim 21 wherein said free end of said first arm is arranged in spaced-apart confronting relation to said free end of said second arm thereby forming a gap between said free ends such that the distance from said free end of said first arm to said first axis of symmetry is different than the distance from said free end of said second arm to said first axis of symmetry.

26. A heart valve according to claim 21 wherein the difference in said distances from said free ends of said arms to said first axis of symmetry is in the range from about between about 0.0125 to about 0.0625 times an internal diameter of said support ring.

27. A heart valve according to claim 21 wherein said free end of said first arm is arranged in spaced-apart confronting relation to said free end of said second arm thereby forming a gap between said free ends such that the distance from said free end of said first arm to said second axis of symmetry is different than the distance from said free end of said second arm to said second axis of symmetry.

28. A heart valve according to claim 27 wherein the difference in said distances from said free ends of said arms to said second axis of symmetry is in the range from about between about 0.0125 to about 0.0625 times an internal diameter of said support ring.

29. A method for operation for a heart valve prosthesis comprising;
(A) rotating an occluder-disc open within a support ring about a first axis so as to allow blood to flow through a fluid passageway of said support ring; and
(B) rotating said occluder-disc closed about said first axis while also incrementally rotating said occluder disc about a second axis so as to shift the portions of said occluder-disc that impact said support ring upon closure of said heart valve.

30. A method for operation for a heart valve prosthesis comprising;
(A) rotating an occluder-disc open within a support ring about a first axis so as to allow blood to flow through a fluid passageway of said support ring; and
(B) rotating said occluder-disc closed about said first axis so that portions of said occluder-disc impact said support ring upon closure of said heart valve at different times.

31. A method for operation for a heart valve prosthesis comprising;
(A) rotating an occluder-disc open within a support ring about a first axis so as to allow blood to flow through a fluid passageway of said support ring; and
(B) rotating said occluder-disc closed about said first axis while also incrementally rotating said occluder disc about a second axis so as to shift the portions of said occluder-disc that impact said support ring upon closure of said heart valve and so that said portions of said occluder-disc that impact said support ring upon closure of said heart valve do so at different times.
